(19) United States
(12) Patent Application Publication
(10) Pub. No.: US 2007/0038297 A1
(43) Pub. Date: Feb. 15, 2007

(54) Medical implant with reinforcement mechanism

(76) Inventors: Donald E. Bobo JR., Santa Ana, CA (US); George Bakis, La Habra Heights, CA (US); Minh T. Ma, Santa Ana, CA (US)

Correspondence Address: EDWARDS LIFESCIENCES CORPORATION, LEGAL DEPARTMENT, ONE EDWARDS WAY, IRVINE, CA, 92614, US

(21) Appl. No.: 11/502,879

(22) Filed: Aug. 11, 2006

Related U.S. Application Data
(60) Provisional application No. 60/707,926, filed on Aug. 12, 2005.

(51) Int. Cl. A61F 2/24

(52) U.S. Cl. 623/2.36; 606/151

(57) ABSTRACT

An improved medical implant for treating mitral regurgitation is provided. The medical implant comprises proximal and distal anchors connected by a bridge. The medical implant is configured to be delivered into a coronary sinus using a minimally invasive procedure. The bridge is preferably made of a shape memory material which is biased to contract after the implant is delivered. The medical implant further comprises a reinforcement mechanism configured to limit stresses and strains along the length of the bridge. In a preferred embodiment, the reinforcement mechanism is fixed to a plurality of attachment points along the bridge, thereby preventing excessive elongation between any two attachment points. A resorbable material is preferably disposed within gaps along the length of the bridge to temporarily maintain the bridge in an elongated condition. After the proximal and distal anchors are secured in the coronary sinus, the resorbable material gradually resorbs, thereby creating tension in the bridge which applies a force along the mitral valve annulus. The reinforcement mechanism ensures that stresses and strains and distributed evenly while the bridge is in tension.

### RELATED APPLICATIONS

The present application claims priority under 35 U.S.C. §119(e) to U.S. Provisional Application No. 60/707,926, filed on Aug. 12, 2005, the entirety of which is hereby incorporated by reference.

### FIELD OF THE INVENTION

The present invention relates to medical implants, and more particularly to medical implants configured for treating mitral valve regurgitation.

### BACKGROUND

The mitral valve is located between the left atrium and left ventricle of the heart. Mitral regurgitation, or leakage from the outflow to the inflow side of the mitral valve, is the most common type of heart valve insufficiency. Mitral regurgitation becomes chronic when the condition persists rather than occurring for only a short time period. Any disorder that weakens or damages the mitral valve may prevent it from closing properly, causing this type of leakage. In many cases, mitral regurgitation is caused by changes in the geometric configurations of the left ventricle, papillary muscles and mitral annulus. These geometric alterations result in incomplete leaflet coaptation during ventricular systole, thereby producing regurgitation.

In recent years, several new minimally invasive techniques have been developed for repairing mitral valves without opening the chest or requiring cardiopulmonary by-pass. At least one of these techniques involves introducing an implant (i.e., endovascular device) into the coronary sinus for reshaping the mitral annulus. The coronary sinus is a blood vessel commencing at the coronary sinus ostium in the right atrium and passing through the atrioventricular groove in close proximity to the posterior, lateral and medial aspects of the mitral annulus. Because of its position adjacent to the mitral annulus, the coronary sinus provides an ideal conduit for positioning an implant to press against the mitral annulus.

In one configuration, an implant for treating mitral regurgitation includes a proximal anchor, a distal anchor, and a bridge extending between the proximal and distal anchors. When the proximal and distal anchors are fixed within the coronary sinus, the bridge portion of the implant applies a compressive force along a posterior region of the mitral valve annulus. The compressive force reshapes the mitral annulus for improving coaption of the mitral valve leaflets. Although it has been found that implants of this type are effective in treating mitral regurgitation, there is a need for an improved device having enhanced structural integrity while maintaining a low profile in the coronary sinus. The present invention addresses this need.

### SUMMARY OF THE INVENTION

Embodiments of the present invention provide an improved device and method for treating mitral regurgitation in a minimally-invasive manner. Certain embodiments provide an improved implant which is configured for deployment partially or entirely within a coronary sinus. The improved implant is preferably formed with a composite structure wherein a reinforcement mechanism is combined with a metallic member. The reinforcement mechanism enhances the structural integrity of the implant by reducing or eliminating undesirable stresses and strains in the metallic member. The reinforcement mechanism also improves the efficacy and controllability of the implant during use. The implant is preferably configured to provide a low profile after deployment in the coronary sinus. The implant is also preferably configured to accelerate tissue ingrowth for enhanced anchoring after deployment.

In one preferred embodiment of the present invention, a medical implant for treating mitral regurgitation comprises a proximal anchor, a distal anchor, and an elongate bridge formed of a shape memory material, wherein the elongate bridge extends between the proximal and distal anchors. The medical implant may be delivered with the bridge in a stretched length; however, the bridge is biased to return towards a shorter, relaxed length. A reinforcement mechanism is attached to the bridge at a plurality of attachment locations. In an important feature, the reinforcement mechanism relieves strain by preventing localized stretching of the bridge. The reinforcement mechanism preferably does prevent contraction of the bridge and therefore does not adversely affect the therapeutic function of the implant. The medical implant is sized for deployment at least partially within a coronary sinus and is configured to apply a compressive force along a posterior portion of the mitral annulus.

The reinforcement mechanism preferably comprises a substantially inelastic material that exhibits little or no stretching while in tension. As a result, the reinforcement mechanism constrains the maximum separation between adjacent attachment points along the medical implant and relieves peak strain. The reinforcement mechanism is preferably attached to the bridge at selected locations such that the bridge will not be damaged or fatigued due to undesirable localized stretching. Accordingly, the reinforcement mechanism provides a limiting member which ensures that the structural integrity of the bridge will not be compromised during use. Furthermore, the reinforcement mechanism provides a redundant attachment mechanism which prevents complete separation in the event of a structural failure.

The reinforcement mechanism may be attached by any suitable means including, but not limited to, tying, gluing, and bonding. Preferred materials for the reinforcement mechanism include nylon, polypropylene, polyethylene, and PET polyester. In one preferred embodiment, the reinforcement mechanism comprises a fiber thread. The fiber thread is preferably a multifilament elongate member; however, a monofilament member may also be used. In another preferred embodiment, the reinforcement member comprises a tubular member surrounding at least a portion of the bridge. The tubular member is preferably made of PET polyester, such as Dacron®.

In another embodiment, a medical implant is provided wherein the reinforcement mechanism extends from the bridge into the proximal and distal anchors of the medical implant. The reinforcement mechanism enhances the attachment of the proximal and distal anchors to the bridge. The reinforcement mechanism preferably extends into the anchors in a manner sufficient to distribute forces evenly along the anchors and thereby avoid stress concentrations.

In another embodiment, a medical implant is provided wherein the reinforcement mechanism provides the primary or only attachment means for connecting the anchors to the bridge. In this configuration, the reinforcement mechanism replaces the metal links between the bridge and the anchors. The bridge and anchors may be manufactured as separate components which are secured together by the reinforcement mechanism. This configuration advantageously eliminates the existence of stress concentrations in the metal links between the bridge and anchors. This configuration may also provide greater anchor flexibility and may comprise a portion of a modular system wherein anchors may be attached to a bridge as desired for a particular application.

In another embodiment, a medical implant has proximal and distal anchors, a shape memory bridge, and a reinforcement mechanism. In this embodiment, the medical implant further comprises a bioresorbable material for temporarily maintaining the shape memory bridge in a stretched length. The bioresorbable material is disposed within gaps or voids in the bridge. As a result, the bridge comprises a shape-changing member that is temporarily held at a stretched length and is biased towards a shorter, relaxed length. As the material is resorbed by the body, the gaps close and the bridge contracts in length. Because the proximal and distal anchors are secured within the coronary sinus, as the bridge contracts towards the relaxed length, tension in the bridge increases. The tension in the bridge produces a compressive force which pushes inward along a posterior portion of the mitral annulus. In this embodiment, the reinforcement mechanism advantageously ensures that the implant transforms to the relaxed length in a desirable manner wherein localized stresses and strains are limited.

In another embodiment, a medical implant having a composite structure comprises a shape memory material and a limiting member attached to the shape memory material. The limiting member is attached to the shape memory material along a plurality of attachment points for limiting the movement between adjacent attachment points. The limiting member provides enhanced controllability over the final shape of the shape memory material. The limiting member is particularly advantageous wherein it is desirable for an implant to transform into a specific shape. The limiting member may further provide a redundant connection between adjacent attachment points. In one preferred configuration, the limiting member comprises at least one fiber thread.

Other objects, features, and advantages of the present invention will become apparent from a consideration of the following detailed description.

### BRIEF DESCRIPTION OF THE DRAWINGS

FIG. 1 is a three-dimensional view of the mitral valve and coronary sinus.

FIG. 2 is a side view of an implant including a proximal anchor, a distal anchor and a bridge connecting the proximal and distal anchors, wherein a resorbable thread is woven into the bridge.

FIG. 3 is an enlarged plan view illustrating a section of the bridge of FIG. 2 wherein a portion of the bridge is held in a stretched condition by the resorbable thread and another portion of the bridge is in a relaxed condition.

FIG. 4 is a side view illustrating an implant including a reinforcement mechanism in the form of a tubular body which extends along the bridge of the implant.

FIG. 5 is plan view of a section of the bridge illustrating preferred attachment points wherein the reinforcement mechanism of FIG. 4 is secured to the bridge.

FIG. 6 is a plan view of a section of a bridge illustrating another preferred embodiment of a reinforcement mechanism comprising a fiber fixed to attachment points along a central region of the bridge.

FIG. 7 is a plan view of a section of a bridge illustrating another preferred embodiment of a reinforcement mechanism comprising two fibers extending along substantially parallel paths and which are fixed to attachment points along opposite sides of the bridge.

FIG. 8 is a plan view illustrating a variation of the embodiment of FIG. 7, wherein additional reinforcement is provided along the end portions of the bridge for enhancing the structural integrity of the implant along regions of high stress concentrations.

FIG. 9 is a plan view of a section of a bridge illustrating yet another preferred embodiment of a reinforcement mechanism comprising two fibers disposed in an interlaced crossing pattern along the length of the bridge.

FIG. 10 is a plan view of a bridge illustrating yet another preferred embodiment of a medical implant which does not include a biodegradable material and wherein a reinforcement mechanism extends along the length of the bridge.

FIG. 11 is a plan view of a section of a bridge illustrating another preferred embodiment of a reinforcement mechanism comprising two fibers fixed to attachment points located on every other expandable cell and wherein the two fibers are attached in a staggered pattern.

FIG. 12 is a plan view illustrating a distal end portion of a medical implant wherein a reinforcement mechanism extends between the bridge portion and the expandable distal anchor.

FIG. 13 is a plan view illustrating a distal end portion of a medical implant wherein a reinforcement mechanism is configured for attaching the bridge to the distal anchor.

### DETAILED DESCRIPTION

Various embodiments of the present invention depict medical devices and methods of use that are well-suited for treating mitral valve regurgitation. However, it should be appreciated that the principles and aspects of the embodiments disclosed and discussed herein are also applicable to other devices having different structures and functionalities. For example, certain structures and methods disclosed herein may also be applicable to other medical devices. In particular, certain structures and methods disclosed herein may be applicable to various other types of medical devices made from shape memory materials. Furthermore, certain embodiments may also be used in conjunction with other medical devices or other procedures not explicitly disclosed. The manner of adapting the embodiments described herein to various other devices and functionalities will become apparent to those of skill in the art in view of the description that follows.

As used herein, “distal” means the direction of a device as it is being inserted into a patient's body or a point of reference closer to the leading end of the device as it is inserted into a patient's body. Similarly, as used herein “proximal” means the direction of a device as it is being removed from a patient's body or a point of reference closer to a trailing end of the device as it is inserted into a patient's body.

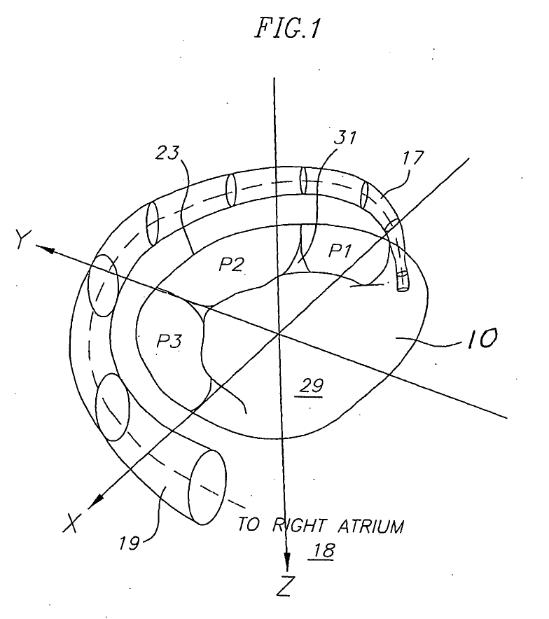

With reference now to FIG. 1, a three-dimensional view of a mitral valve 10 and a coronary sinus 17 is shown. From this view, it can be seen that the coronary sinus extends around a posterior region of the mitral valve 10. The coronary sinus is a relatively large vessel that receives venous drainage from the heart muscle. Blood flows through the coronary sinus and empties into the right atrium 18 through a coronary ostium 19. A mitral annulus 23 is a portion of tissue surrounding a mitral valve orifice to which the valve leaflets attach. The mitral valve 10 has two leaflets, an anterior leaflet 29 and a posterior leaflet 31. The posterior leaflet has three scallops P1, P2 and P3. As used herein, the term coronary sinus 17 is used as a generic term that describes the portion of the vena return system that is primarily situated adjacent to the mitral valve 10 and extends, at least in part, along the atrioventricular groove. Accordingly, the term coronary sinus 17 includes the great cardiac vein and all other related portions of the vena return system.

Dilation of the mitral valve annulus 23 is the primary cause of regurgitation through the mitral valve 10. More particularly, when a posterior aspect of the mitral annulus 23 dilates, one or more of the posterior leaflet scallops P1, P2, P3 moves away from the anterior leaflet 29. As a result, the anterior and posterior leaflets of the mitral valve fail to close completely during ventricular systole and blood flows backward (i.e., regurgitates) through the resulting gap. To reduce or eliminate mitral regurgitation, it is desirable to move the posterior aspect of the mitral annulus 23 in an anterior direction, thereby narrowing or closing the gap between the leaflets.

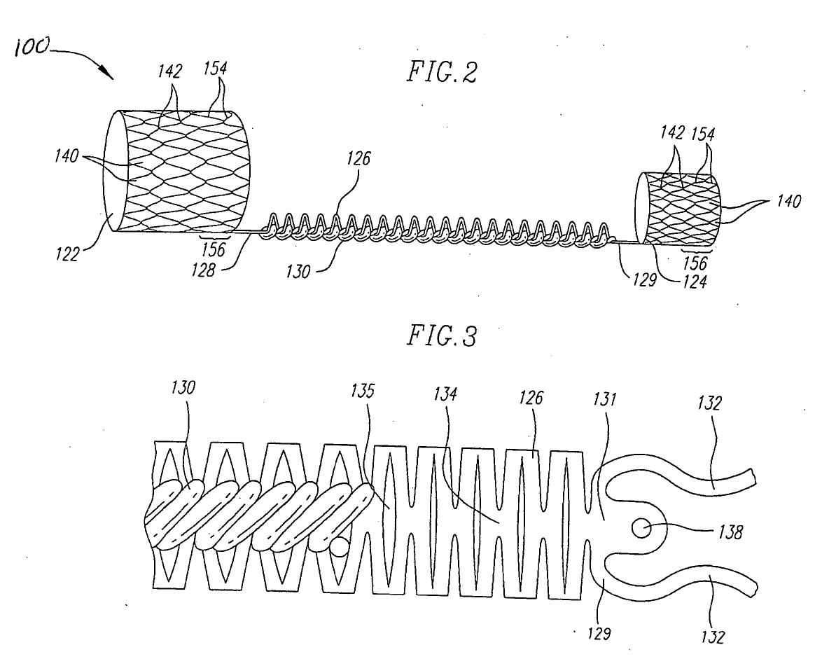

With reference now to FIGS. 2 and 3, one embodiment of a mitral valve repair implant 100 is illustrated. The implant is sized for deployment in the coronary sinus and is configured to apply a compressive force along the posterior portion of the mitral annulus. As illustrated, the implant 100 includes a proximal anchor 122 and a distal anchor 124 connected by a bridge 126. The bridge 126 is configured to foreshorten after the proximal and distal anchors are secured within the coronary sinus. A resorbable material is disposed within openings 135 in the bridge.

Resorbable materials are those that, when implanted into a human body, are resorbed by the body by means of enzymatic degradation and also by active absorption by blood cells and tissue cells of the human body. Examples of such resorbable materials are PDS (Polydioxanon), Pronova (Poly-hexafluoropropylen-VDF), Maxon (Polyglyconat), Dexon (polyglycolic acid) and Vicryl (Polyglactin). As explained in more detail below, a resorbable material may be used in combination with a shape memory material, such as Nitinol, Elgiloy or spring steel to allow the superelastic material to return to a predetermined shape over a period of time.

The resorbable material maintains the bridge in a stretched length during delivery and deployment. Over time, the resorbable material is resorbed and the bridge returns to its relaxed (i.e., shortened) length. As the bridge shortens, it tightens against the posterior aspect of the mitral annulus for reducing dilation of the mitral annulus. Additional details regarding medical implants and preferred methods of use for treating mitral valve regurgitation may be found in Assignee's U.S. Pat. No. 6,210,432, U.S. Pat. No. 6,997,951, U.S. Pat. No. 7,090,695, U.S. application Ser. No. 10/141,348, filed May 9, 2002, and U.S. application Ser. No. 11/238,853, filed Sep. 28, 2005, each of which is hereby incorporated by reference in its entirety.

With continued reference to the embodiment illustrated in FIG. 2, in one preferred construction, the proximal and distal anchors 122, 124 are both preferably cylindrical in shape and are formed from tubes of shape memory material, such as, for example, Nitinol. However, the anchors 122, 124 may also be made from any other suitable material, such as, for example, stainless steel. In the illustrated embodiment, both anchors 122, 124 have a mesh configuration comprising loops 154 of zig-zag shaped shape memory material having alternating peaks 142. The loops 154 are connected at each peak 142 to form rings 156 of four-sided openings 140. It will be appreciated that, although one particular type of anchor mechanism is shown for purposes of illustration, a wide variety of anchoring mechanisms may be used for securing the implant within the coronary sinus.

Each of the proximal and distal anchors 122, 124 has a compressed state and an expanded state. In the compressed state, the anchors 122, 124 have a diameter that is less than the diameter of the coronary sinus 17. In the compressed state, the anchors 122, 124 have a substantially uniform diameter of between about 1.5 mm and 4 mm. In the expanded state, the anchors 122, 124 have a diameter that is preferably about equal to or greater than a diameter of the section of a non-expanded coronary sinus 17 to which each anchor will be aligned. Since the coronary sinus 17 has a greater diameter at its proximal end than at its distal end, in the expanded state the diameter of the proximal anchor 122 is preferably between about 10 mm and 18 mm and the diameter of the distal anchor 124 is preferably between about 3 mm and 8 mm.

The bridge 126 is preferably connected to the proximal anchor 122 and distal anchor 124 by links 128, 129. More specifically, as shown in FIG. 2, a proximal link 128 connects the proximal anchor 122 to a proximal end of the bridge 126 and a distal link 129 connects the distal anchor 124 to a distal end of the bridge 126. In the illustrated embodiment, each of the links 128,129 has a base 131 and arms 132 that extend from the base. The arms are connected to peaks 142 on each anchor 122, 124. Further, the links 128, 129 may be provided with a hole 138, as shown in FIG. 3, which serves as a means through which to pass an end of the resorbable thread and secure it to the bridge 126.

With continued reference to the embodiment illustrated in FIGS. 2 and 3, the bridge 126 is preferably formed with a plurality of expandable elements (or cells) 134. In the illustrated embodiment, each expandable element 134 generally comprises an X-shaped member, wherein each X-shaped member is connected to an adjacent X-shaped member at the extremities of the “X.” The connection of the X-shaped members creates a plurality of openings 135 between the expandable elements 134. As best shown in FIG. 3, the openings are larger when the bridge is in a stretched condition. If desired, the X-shaped members may be formed with rounded edges that minimize the chance that a sharp edge of the bridge 126 will damage the coronary sinus 17 during delivery of the implant 100.

In the illustrated embodiment, the resorbable thread 130 is woven into the openings 135 (as shown in FIGS. 2 and 3) between adjacent expandable elements 134. The thread acts as a temporary spacer which prevents the openings from contracting. Accordingly, the thread temporarily maintains the bridge 126 in its stretched condition. As the resorbable thread 130 dissolves over time, the openings 135 contract (i.e., become more narrow in width). As a result, the bridge gradually reduces in length and pulls on the proximal and distal anchors. Because the proximal and distal anchors are secured within the coronary sinus, the reduction in bridge length creates tension in the bridge. When the implant is initially deployed, the bridge follows a curved path (i.e., a path which follows the curvature of the coronary sinus). However, the resulting tension causes the bridge to adjust toward a straighter path. As the shape of the bridge straightens, the bridge applies a compressive force along the posterior portion of the mitral valve annulus, thereby reshaping the mitral valve annulus and reducing mitral regurgitation.

Although it has been determined that medical implants of this type are effective in treating mitral valve regurgitation, the tension created in the bridge during foreshortening may result in high stress concentrations, such as along the links 128, 129 wherein the bridge attaches to the anchors. Furthermore, although the total length of the bridge is reduced as the resorbable material is resorbed, it is possible that the increased tension can lead to localized regions of stretching or stress along the bridge. This is an undesirable effect because high stresses and strains can compromise the structural integrity of the implant 100. Accordingly, a need exists for an improved medical implant that is configured to foreshorten without being subjected to high localized stresses or strains. As will be discussed in more detail below, this need is addressed by an improved medical implant having a composite structure wherein a reinforcement mechanism is combined with a shape memory material to regulate the transformation of the implant after delivery into the body.

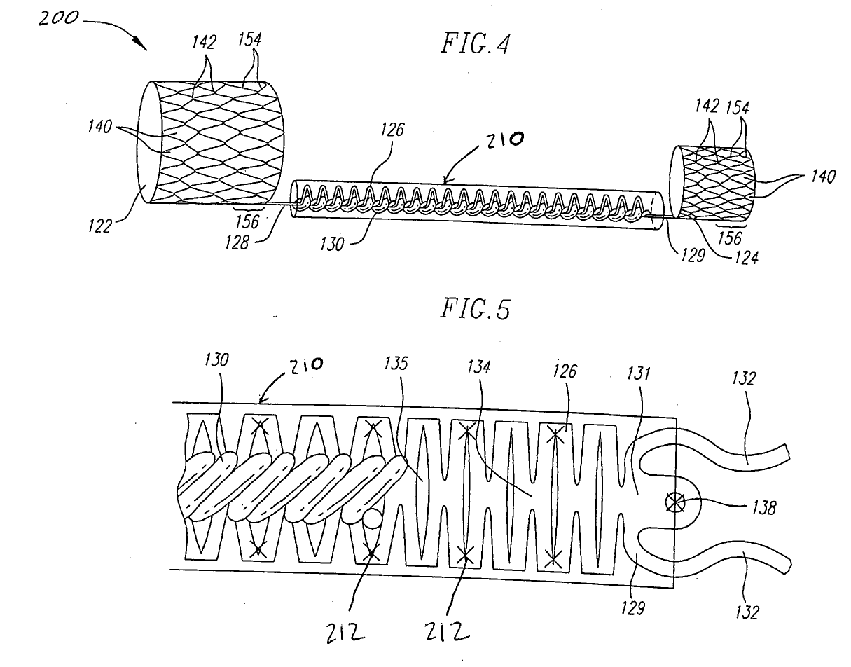

With reference now to FIG. 4, for purposes of illustration, an improved mitral valve repair implant 200 will be described in accordance with one preferred embodiment of the present invention. The implant is preferably formed at least in part of a shape memory material and is configured to be used for treating mitral regurgitation as generally described above. Although the implant is described with respect to treating mitral valves, the features of the implant may also be applied to other treatments, such as treatment of the tricuspid valve.

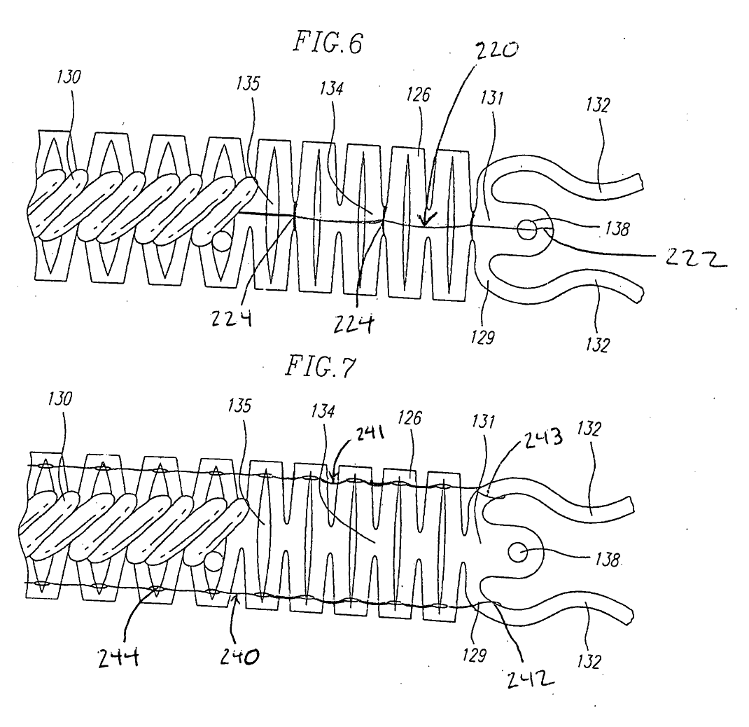

The implant comprises a proximal anchor 122, a distal anchor 124 and a bridge 126 formed of a shape memory material. A resorbable material 130 is disposed along the bridge to temporarily maintain the bridge in a stretched condition. The improved implant 200 further comprises a reinforcement mechanism 210, or limiting member, configured to reduce localized stresses and strains on the bridge 126 while the bridge foreshortens during use. As illustrated in FIG. 5, the reinforcement mechanism is secured to the bridge along a plurality of attachment points 212 to regulate contraction of the bridge. The reinforcement mechanism may be made of a fabric or other suitable material configured to exhibit little or no stretching. In certain preferred embodiments, the reinforcement mechanism is formed at least in part of a material that encourages tissue ingrowth into the bridge portion of the implant. For example, the reinforcement mechanism may be formed of an abrasive material or a biologically active material that accelerates tissue growth. It will be recognized that tissue ingrowth further anchors the implant within the coronary sinus and thereby improves the effectiveness of the treatment.

In preferred embodiments, the reinforcement mechanism provides a “stretch limiter” which constrains or limits the maximum separation between adjacent attachment points on the bridge and thereby eliminates the possibility of undesirable localized stretching. The elimination of localized stretching ensures that the strain is distributed in a substantially even manner along the bridge. Accordingly, the reinforcement mechanism advantageously reduces metal fatigue and increases the design life of the medical implant 200. In certain preferred embodiments, the reinforcement mechanism is configured to prevent the adjacent elements from being stretched beyond the initial delivery condition which may be, for example, about 150% of the relaxed length. Furthermore, the reinforcement mechanism may provide a safety device which prevents separation in the event of a structural failure. This is an advantageous feature because shape memory materials, and most metals, can exhibit structural fatigue when exposed to a large number of stress cycles, as may occur after placement in a coronary sinus. Accordingly, in preferred embodiments, the reinforcement mechanism helps distribute forces, relieves strain and provides a redundant attachment member for enhancing the structural integrity of the device. Still further, the reinforcement mechanism may facilitate the manufacture of the implant by limiting the stretching between adjacent expandable elements to the desired separation while the resorbable material 130 is applied within the gaps 135.

With continued reference to FIG. 4, one preferred embodiment of the implant 200 includes a reinforcement mechanism in the form of an elongate tube or sock 210 which extends along at least a portion of the bridge. The reinforcement mechanism may be made of any suitable biocompatible material, such as, for example, Dacron®. FIG. 5 provides a plan view along a portion of the bridge wherein it can be seen that the reinforcement mechanism is attached to the bridge at a plurality of attachment points 212. The reinforcement mechanism is preferably stitched or tied to the bridge using suture or fiber. In the illustrated embodiment, an attachment point is provided on every other expandable element 134. However, in alternative configurations, the reinforcement mechanism could be attached at any desired locations. Furthermore, the reinforcement mechanism may be secured by any other suitable means, such as glued, looped or otherwise attached to the bridge. Because the reinforcement mechanism does not stretch, the reinforcement mechanism is configured to prevent excessive separation between adjacent expandable elements. At the same time, the reinforcement mechanism allows the bridge to contract and does not interfere with the therapeutic function of the implant.

With reference now to FIG. 6, a portion of a bridge is illustrated wherein a reinforcement mechanism takes the form of an elongate fiber 220. In one preferred embodiment, the fiber is a non-biodegradable polymer fiber that is intertwined with the segments of the bridge. The fiber is preferably applied to the implant when the bridge is in a stretched configuration. For example, in one preferred embodiment, the fiber is applied while the bridge is stretched to a length of about 150% of its relaxed length. In preferred embodiments, the fiber may be a multi-filament, monofilament, braided, coated, extruded, and/or molded elongate member. Preferred materials include polymers, co-polymers, fabric having a high fatigue, cycle and/or flexure characteristics. With continued reference to FIG. 6, the fiber is fixed to attachment points 224 along the central region of the bridge. In the illustrated embodiment, the fiber 220 has at least one end 222 which extends through the hole 138 in the link 129 for securement to the link. Fabrication methods include: threaded with a knot, machine sewn with eyelets, applied with adhesive, and temperature set-molded.

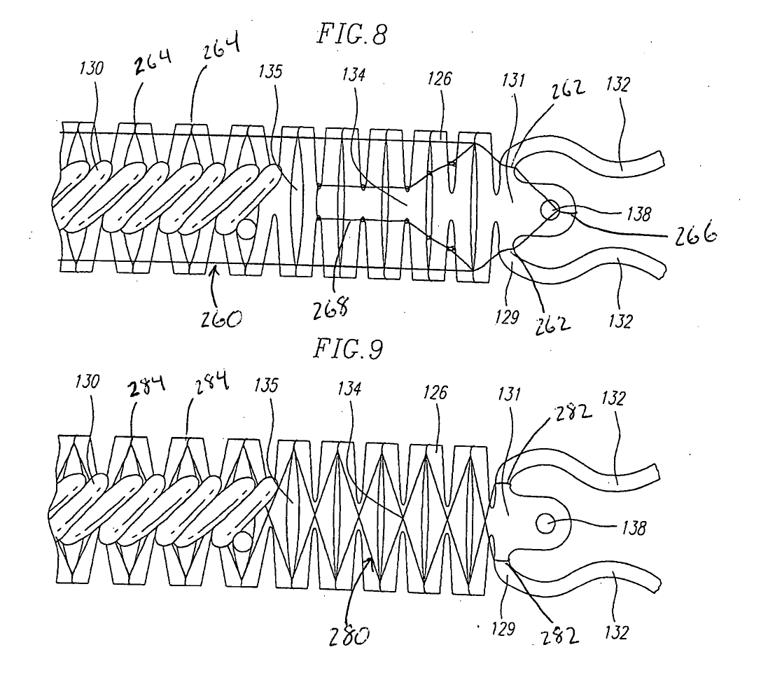

With reference to FIG. 7, a portion of a bridge is illustrated wherein a reinforcement mechanism 240 comprises a pair of elongate fibers extending along the length of the bridge. In this embodiment, the fibers 240 are fixed to an attachment point 244 on each expandable element 134. One end of each fiber 242, 243 is preferably tied to the link 129. The fibers may be attached to the bridge by a variety of means, such as, for example, by knots, weaving, adhesives, thermal bonding, or any other appropriate attachment mechanism. In other embodiments, the bridge may be manufactured with eyelets or holes configured to capture and retain a portion of the fiber.

With reference to FIG. 8, a portion of a bridge is illustrated wherein a reinforcement mechanism 260 is similar to that described above with reference to FIG. 7; however, this embodiment includes additional reinforcement along the end portions of the bridge. The fibers are fixed to an attachment point 264 on each expandable element 134. A portion of the fiber 262 is also tied to the link 129 with another portion 266 extending through the hole 138. Still further, the fiber includes an end portion 268 which wraps back around and is attached once again to the bridge. The end portion 268 provides additional reinforcement along the end portion of the bridge and along the attachment points (i.e., links 128, 129) wherein the bridge connects to the anchors.

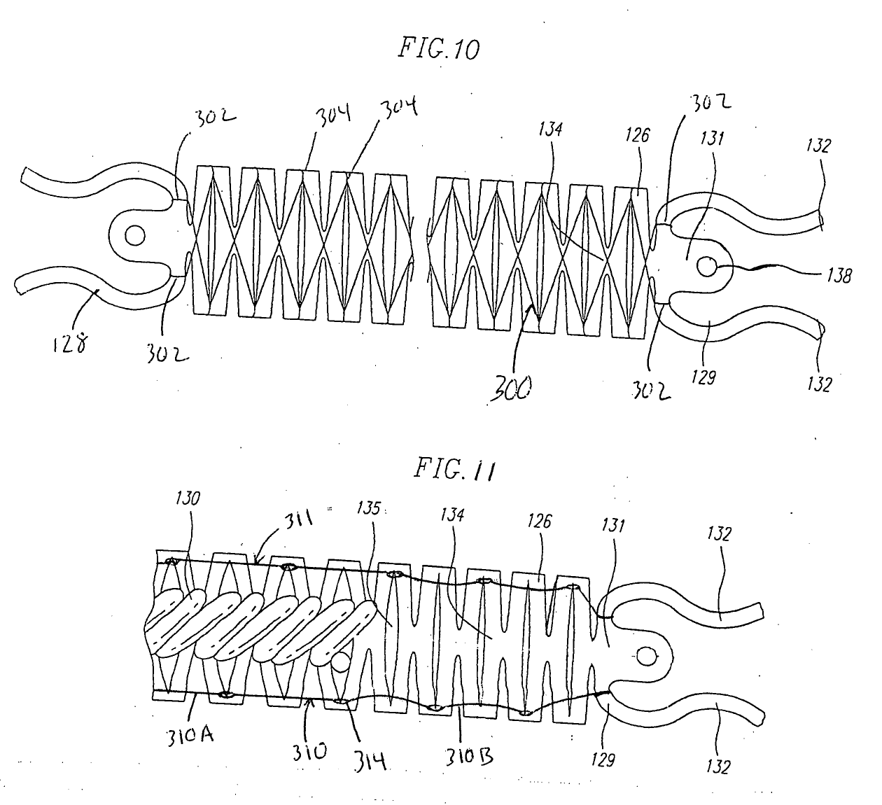

With reference to FIG. 9, a portion of a bridge is illustrated wherein a reinforcement mechanism 280 comprises a pair of elongate fibers extending along the length of the bridge in an interlaced arrangement. In this embodiment, the fibers 280 are fixed to an attachment point 284 on each expandable element 134. The fiber ends 282 are tied to the link 129. In this embodiment, the fibers 280 are attached to the bridge 126 in a crossing pattern which does not interfere with the application or resorbtion of the bioresorbable material. As discussed above, the primary purpose of the fibers 280 is to limit the amount of separation between adjacent attachment points 284 along the bridge. In other words, the fibers 280 provide a reinforcement mechanism that prevents undesirable stretching of the implant while the bridge is in tension. As a result, the implant is capable of applying a compressive force along the posterior portion of the mitral valve annulus without compromising the structural integrity of the implant. Furthermore, the fiber 280 does not interfere with the contraction of the bridge over time.

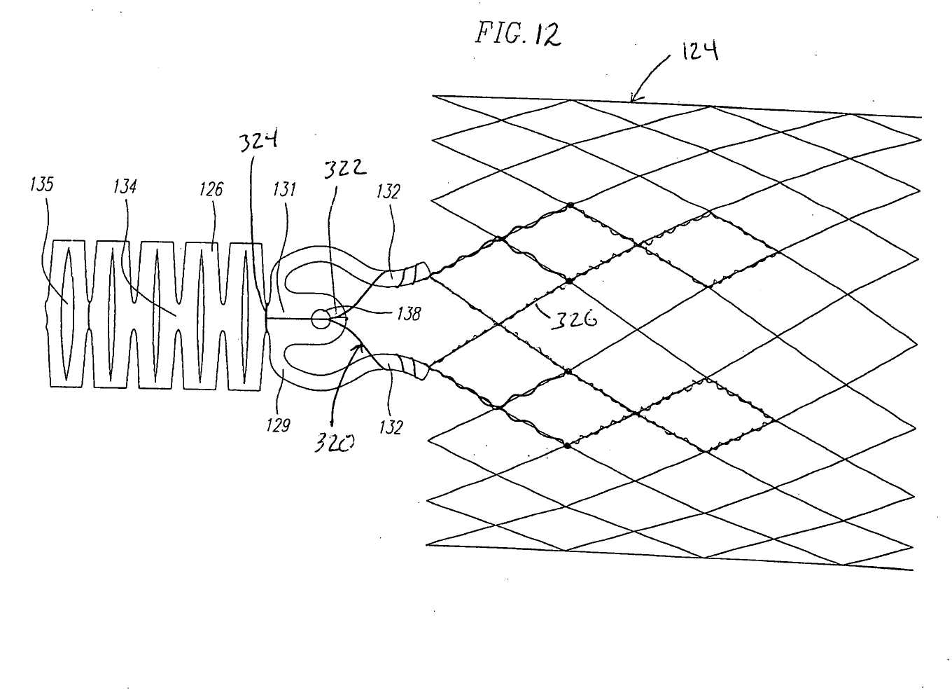

With reference to FIG. 10, a portion of a bridge is illustrated which does not include a bioresorbable material. Rather, this embodiment is configured to act upon the mitral valve annulus in an acute manner. In this embodiment, the bridge is preferably formed of a shape memory material and may have a configuration including X-shaped cells, similar to that described above. Similar to the embodiment described above with respect to FIG. 9, the bridge further preferably comprises a reinforcement mechanism which limits stretching of the bridge during deployment and provides a safety mechanism to prevent separation in the event of a fracture. In this embodiment, the reinforcement mechanism preferably extends along the entire length of the bridge and has ends 302 which are attached to the links 128, 129.

Because the embodiment illustrated in FIG. 10 does not include a bioresorbable material, the method of using the implant preferably differs from the above described implants. More particularly, the implant is used to acutely treat the mitral valve. The method generally comprises deploying the distal anchor in the coronary sinus and pulling the proximal anchor in a proximal direction to create tension in the bridge for applying a compressive force along the posterior portion of the mitral valve annulus. As the proximal anchor is pulled, the bridge 126 stretches as a function of the pulling force. After the mitral valve annulus has been sufficiently reshaped, the proximal anchor is deployed, preferably at a location within or adjacent to the coronary ostium. In this embodiment, the reinforcement mechanism 300 ensures that the bridge 126 is stretched evenly along its length during deployment. Furthermore, as discussed above, the reinforcement mechanism provides a redundant attachment mechanism which eliminates the possibility of separation along the bridge resulting from structural fatigue. After deployment, the tension in the bridge ensures that a continuous compressive force is applied along the mitral valve annulus. Furthermore, over time, the bridge may foreshorten as the mitral valve annulus is reshaped and the bridge strives to return to its relaxed length.

With reference to FIG. 11, a portion of a bridge is illustrated wherein a reinforcement mechanism comprises a pair of elongate fibers 310, 311 extending along the length of the bridge. In this embodiment, the fibers 310, 311 are fixed to attachment points 314 on every other expandable element 134. The fibers are preferably tied to a link 129. The fibers may be attached to the bridge by a variety of means, such as, for example, by knots, weaving, adhesives, thermal bonding, or any other appropriate attachment mechanism. To further distribute the load, the fibers 310, 311 are attached to the bridge in a staggered pattern such that only one fiber is attached to each element. For purposes of illustration, it can be seen that a first portion 310A of the first fiber 310 is in tension because the adjacent portion of the bridge is in a stretched condition. A second portion 310B of the first fiber 310 is not in tension and has some slack because the second portion is attached to a different portion of the bridge that is in a relaxed (i.e., unstretched) condition.

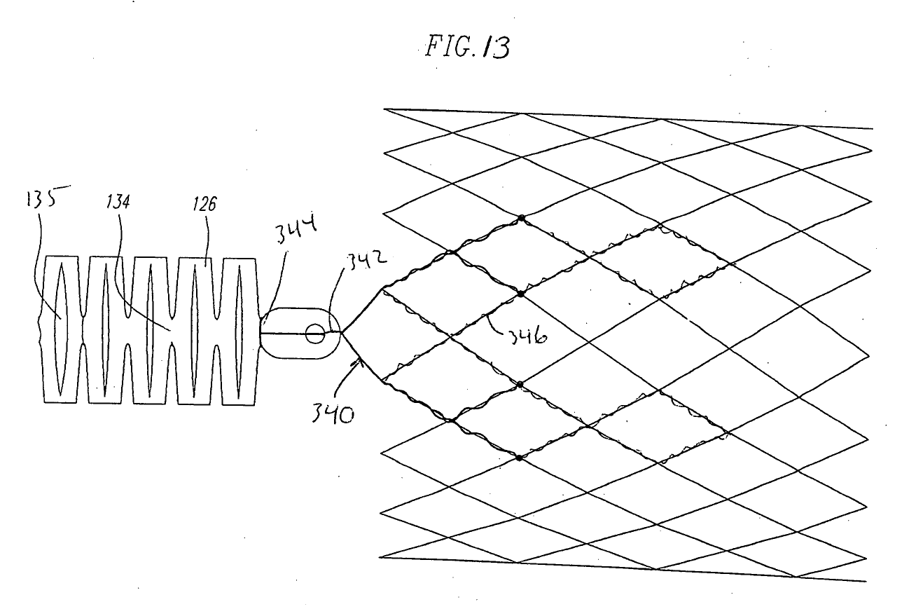

With reference now to FIG. 12, a distal end portion of a medical implant is illustrated wherein a reinforcement mechanism 320, preferably in the form of a fiber, extends from the bridge 126 into the distal anchor 124. The reinforcement mechanism 320 may extend only between the bridge and distal anchor, as shown, or may continue along the length of the implant for reinforcing the bridge. Although reinforcement is only illustrated along the distal end portion of the medical implant, it may be desirable to include a similar reinforcement mechanism along the proximal end portion.

With reference now to FIG. 13, a distal end portion of a medical implant is illustrated wherein the bridge and distal anchor are separate components. In this embodiment, the distal anchor (and preferably the proximal anchor) is attached to the bridge using an attachment mechanism 320, such as, for example, a suture line or fiber. In one example, the implant is configured to allow the anchor to pivot relative to the bridge in a substantially unrestrained manner. Because the bridge 126 and anchor 124 are separate components, no stress concentrations will occur along the connection between the bridge and anchor. For example, an anchor and a bridge may assume different orientations in the coronary sinus without bending or kinking a portion of the implant. This advantageous reduces stress concentrations at attachment points on the implant.

In the above discussion, some medical implants have been described which include a bioresorable material, while others do not include a bioresorbable material. It will be appreciated that medical implants may also be provided wherein a bioresorbable material is disposed along only a portion of the bridge. In this “hybrid” embodiment, a portion of the bridge exhibits delayed memory qualities, while the remaining portion of the bridge assumes its final shape at the time of deployment.

In each of the above-described embodiments, the reinforcement mechanism is preferably formed of a material that exhibits little or no stretching under tension. However, in alternative embodiments, a reinforcement mechanism may be provided which exhibits a desirable amount of “limited stretching” to offload a portion of the stress on the bridge. Still further, the reinforcement mechanism may be configured to comprise an elongate member formed of an elastic or shape memory material that provides a force configured to enhance foreshortening of the bridge. In this variation, the reinforcement mechanism may provide a primary or secondary cinching force for creating tension and thereby applying a compressive force along the mitral valve annulus. In yet another variation, the reinforcement mechanism may comprise a hydrophilic material that tightens in vivo. With these and other similar embodiments, it may not be necessary to use a bridge formed of a shape memory material.

Although various embodiments of medical implants for treating mitral regurgitation have been described above for purposes of illustration, it will be appreciated that aspects of the present inventions have a wide variety of alternative applications. For example, it will be appreciated that an implant having a composite structure wherein a shape memory material is combined with a limiting member, such as a reinforcement mechanism, can be used in a wide variety of treatment procedures. The combination of features described herein provides improved controllability over the transformation and final shape of a structure formed entirely or in part with a shape memory material. In other words, the limiting member provides a guide to ensure that the device will transform into a specific desired shape. Furthermore, the combination of features described herein provides a safety mechanism which prevents separation in the event of a structural failure. This may be particularly advantageous for improving the structural integrity of medical devices made of shape memory materials which undergo a large number of stress cycles. Examples of shape memory materials include shape memory metals, such as Nitinol, and shape memory polymers. In addition, it will be appreciated that aspects disclosed herein may also be combined with other elastic or semi-elastic materials to provide a wide variety of reinforced devices while remaining with the scope of the invention. Still further, as discussed above, a biodegradable material may be combined with the limiting member and shape memory material to provide an implant that gradually transforms into a specific shape.

In addition to limiting expansion of a shape memory material, a reinforcement mechanism may be used to limit the amount of contraction of an underlying structure, rather than limiting the stretching. This embodiment would be particularly desirable for providing a shape memory device that contracts to a particular (i.e., specific) shape for treating a patient. In this case, it may be preferable to dispose a substantially rigid member within gaps or spaces along a shape memory device to limit contraction. It will also be appreciated that aspects of the present invention may be combined with implants formed of other biocompatible materials, such as stainless steel or titanium, to provide reinforcement and/or shape control.

In one alternative application, aspects of the present invention are applicable to treating pathological heart growth. A basket formed of a shape memory material may be placed around at least a portion of the heart. Over time, the basket shrinks to constrain the heart and prevent further growth. Reinforcement mechanisms of the type described above are disposed along the basket to enhance structural integrity or to control the transformation of the basket to a specific shape. In a similar approach, a constraining device formed of a shape memory material with a reinforcement mechanism may be used to treat alveoloar sac growth in the lungs. Further details regarding these and other alternative treatment procedure can be found in Applicant's co-pending U.S. application Ser. No. 10/141,348, filed on May 9, 2002. In other applications, aspects of the reinforcement mechanisms disclosed herein may be used with stents, vena cava filters, atrial septal defect closure devices, ventricular septal defect closure devices, patent foramen ovale closure devices and a wide variety of other implantable devices.

Exemplary embodiments of the invention have been described, but the invention is not limited to these embodiments. For example, although particular types of medical implants have been described for purposes of discussion, the improvements disclosed herein may be applicable to wide variety of medical devices while remaining with the scope and spirit of the present invention. Furthermore, various modifications may be made within the scope without departing from the subject matter of the invention described in the description of the invention, and the accompanying drawings.

What is claimed is:

1. A medical implant for treating heart valve pathologies, comprising:
a proximal anchor;
a distal anchor;
an elongate bridge formed of a shape memory material, the elongate bridge extending between the proximal and distal anchors; and
a reinforcement mechanism attached to the bridge at a plurality of attachment locations for limiting a maximum separation between adjacent attachment locations;
wherein the medical implant is sized for deployment at least partially in a blood vessel and the bridge is configured to apply a force along a portion of a valve annulus.

2. The medical implant of claim 1, further comprising a resorbable material disposed along the elongate bridge for maintaining the bridge in a stretched length.

3. The medical implant of claim 1, wherein the reinforcement mechanism comprises a fiber thread.

4. The medical implant of claim 1, wherein the reinforcement mechanism comprises a tubular member surrounding at least a portion of the bridge.

5. The medical implant of claim 4, wherein the tubular member is made of Dacron®.

6. The medical implant of claim 1, wherein the reinforcement mechanism is attached to the proximal and distal anchors.

7. The medical implant of claim 1, wherein the proximal and distal anchors are flexibly attached to the bridge.

8. The medical implant of claim 7, wherein the proximal and distal anchors are attached to the bridge only by the reinforcement mechanism.

9. The medical implant of claim 1, wherein the bridge includes a plurality of expandable elements and wherein the reinforcement mechanism comprises a first fiber attached to every other expandable element.

10. The medical implant of claim 9, wherein the reinforcement mechanism further comprises a second fiber attached to every other element and wherein the fibers are attached in a staggered pattern.

11. A medical implant, comprising:
a shape memory material; and
a reinforcement mechanism attached to the shape memory material along a plurality of attachment points for limiting the movement between adjacent attachment points after delivery into a human body.

12. The medical implant of claim 11, further comprising a resorbable material disposed within gaps along the shape memory material.

13. The medical implant of claim 11, wherein the reinforcement mechanism limits the maximum separation between adjacent attachment points.

14. The medical implant of claim 11, wherein the reinforcement mechanism limits the expansion of the shape memory material.

15. The medical implant of claim 14, wherein the reinforcement mechanism comprises an elongate fiber.

16. The medical implant of claim 14, wherein the reinforcement mechanism is tied to the shape memory material at the attachment points.

17. The medical implant of claim 14, wherein the reinforcement mechanism is glued to the shape memory material at the attachment points.

18. A medical implant having a composite structure, comprising:
a shape memory material; and
a limiting member attached to the shape memory material along a plurality of attachment points for limiting the movement between adjacent attachment points.

19. The medical implant of claim 18, wherein the limiting member further provides a redundant connection between adjacent attachment points.

20. The medical implant of claim 19, wherein the limiting member comprises at least one fiber thread.
